(19) United States
(12) Patent Application Publication
(10) Pub. No.: US 2025/0134959 A1
(43) Pub. Date: May 1, 2025

(54) GRANULOCYTE-MACROPHAGE COLONY-STIMULATING FACTOR-BASED TREATMENTS FOR NEURODEGENERATIVE OR NEUROLOGICAL DISEASES OR DISORDERS

(71) Applicant: Partner Therapeutics, Inc., Lexington, MA (US)

(72) Inventors: Edwin ROCK, Lexington, MA (US); Ila JOSHI, Lexington, MA (US); Timothy BOYD, Lexington, MA (US)

(21) Appl. No.: 18/837,235

(22) PCT Filed: Feb. 10, 2023
(86) PCT No.: PCT/US2023/062374

§ 371 Date: Aug. 9, 2024

Related U.S. Application Data
(60) Provisional application No. 63/309,220, filed on Feb. 11, 2022.

(51) Int. Cl. A61K 38/19; A61K 45/06; G01N 33/68

(52) U.S. Cl. CPC A61K 38/193; A61K 45/06; G01N 33/6896; G01N 2333/4703; G01N 2333/70596; G01N 2800/28; G01N 2800/52

(57) ABSTRACT

The present disclosure relates to the treatment of neurodegenerative or neurological diseases or disorders with granulocyte-macrophage colony-stimulating factor.

### CROSS-REFERENCE TO RELATED APPLICATIONS

This application claims the benefit of U.S. Provisional Patent Application No. 63/309,220, filed Feb. 11, 2022, the entire contents of which are hereby incorporated by reference in their entirety.

### FIELD

This disclosure relates to, in part, treatment and/or mitigation of neurodegenerative or neurological diseases or disorders, as well as diagnostic, prognostic and patient selection methods.

### DESCRIPTION OF THE TEXT FILE SUBMITTED ELECTRONICALLY

This application contains a Sequence Listing in XML format submitted electronically herewith via Patent Center. The contents of the XML copy, created on Feb. 9, 2023, is named “PNR-008PC 127114-5008.xml” and is 8,192 bytes in size. The Sequence Listing is incorporated herein by reference in its entirety.

### BACKGROUND

Neurodegenerative or neurological diseases or disorders are increasingly recognized as major causes of death and disability (including disability-adjusted life-years (DALYs; the sum of years of life lost [YLLs] and years lived with disability [YLDs]) worldwide. Globally, in 2016, neurological disorders were the leading cause of DALYs (˜276 million) and second leading cause of deaths (˜9.0 million). The absolute number of deaths and DALYs from all neurological disorders combined increased (deaths by 39% and DALYs by 15%) whereas their age-standardized rates decreased (deaths by 28% and DALYs by 27%) between 1990 and 2016. The only neurological disorders that had a decrease in rates and absolute numbers of deaths and DALYs were tetanus, meningitis, and encephalitis. The four largest contributors of neurological DALYs were stroke (42.2%), migraine (16.3%), Alzheimer's and other dementias (10.4%), and meningitis (7.9%). These neurological disorders include but are not limited to tetanus, meningitis, encephalitis, stroke, brain and other CNS cancers, traumatic brain injury, spinal cord injury, Alzheimer's disease (AD) and other dementias, amyotropic lateral sclerosis (ALS), Parkinson's disease (PD), the prototypic neuroinflammatory disease multiple sclerosis (MS), Huntington's disease (HD) or Huntington's chorea, motor neuron diseases, idiopathic epilepsy, migraine, tension-type headache, and a residual category for other less common neurological disorders. See Global Burden of Diseases, Injuries, and Risk Factors Study (GBD). Lancet Neurol 2019; 18:459-80.

Neurodegenerative or neurological diseases or disorders can be broadly classified by their clinical presentations, with extrapyramidal and pyramidal movement disorders and cognitive or behavioral disorders being the most common. Few patients have pure syndromes, with most having mixed clinical features. Although neurodegenerative or neurological diseases or disorders are typically defined by specific protein accumulations and anatomic vulnerability, these neurodegenerative or neurological diseases or disorders share many fundamental processes associated with progressive neuronal dysfunction and death, such as proteotoxic stress and its attendant abnormalities in ubiquitin—proteasomal and autophagosomal/lysosomal systems, oxidative stress, programmed cell death, and neuroinflammation. See Dugger B N and Dickson D W. Cold Spring Harb Perspect Biol. 2017. 9(7): a028035.

There is an increasing recognition that inflammation can play a critical role in neurodegenerative or neurological diseases or disorders of the CNS. Adaptive versus innate immune responses have been observed at various stages of neurodegenerative or neurological diseases or disorders. These differential immune responses may not only drive disease processes but could serve as therapeutic targets. Ongoing investigations into the specific inflammatory mechanisms that play roles in disease causation and progression have revealed lessons about inflammation-driven neurodegeneration understanding the advent of these diseases, as well as therapeutics to treat them. An increasing number of immunotherapeutic strategies that have been successful in MS are now being applied to other neurodegenerative or neurological diseases or disorders. Some approaches suppress CNS immune mechanisms, while others harness the immune system to clear deleterious products and cells. See Mamun A A and Liu F. Neurol Neurother. 2017. 2(1); Chitnas T and Weiner H L. J Clin Invest. 2017. 127(10): 3577-3587. Not all immune responses in the CNS are detrimental, and in many cases, they actually aid repair and regeneration. For example, microglia clear debris after myelin damage and when this is impeded, delayed regeneration occurs. Immune activation is also crucial to limit neurotropic viral infections and removes necrotic cells following ischemia. Thus, microglia can exert dual roles in neurodegeneration, both as instigators of damage and as guardians of brain homeostasis. Besides microglia, T cells can also aid recovery during neurodegenerative or neurological diseases or disorders, although the exact mechanisms for this beneficial role of T cells are not clear. Detailed studies of neuroimmune interaction at both cellular and molecular levels have revealed complex interactions, demonstrating that immune cells can secrete both neurotoxic and neuroprotective molecules. As such, regulation of the immune response in neurological disorders has therapeutic value. See Neumann H et al. Brain. 2009. 132:288-95; Schwartz M et al. Neuroscience. 2009. 158:1133-42; Amor S et al. Immunology. 2010. 129(2):154-69.

Since therapeutic response can vary on the basis of heterogeneous clinical and molecular phenotypes, a shift toward personalized or precision medicine approaches, including biomarker development and validation, has been thought to improve the management of many neurodegenerative or neurological diseases or disorders. Substantial progress in molecular immunology, coupled with an increased focus on translational research and personalized medicine, has resulted in a rapid expansion in the field of immune biomarkers in recent years. Such biomarkers might be used as an objective measure of normal versus pathogenic processes or indicator of pharmacological responses to therapeutic inventions. See Biomarkers Definitions Working Group. Clin Pharmacol Ther. 2001. 69(3): 89-95; Willis J C D and Lord G M. Nat Rev Immunol. 2015. 15:323-329; Renert-Yuval Y et al. J Allergy Clin Immunol. 2021. 147(4): 1174-1190.

Cluster of Differentiation-26 (CD26), is a 110 kD cell-surface glycoprotein and a known T-cell activation molecule. CD26 has a known dipeptidyl-peptidase IV (DPP-IV) activity in its extracellular domain. This ecto-enzyme is capable of cleaving amino terminal dipeptides from polypeptides with either L-proline or L-alanine in the penultimate position. On human T cells, CD26 expression appears late in thymic differentiation and is preferentially restricted to the CD4+ helper/memory population, and CD26 can deliver a potent co-stimulatory T-cell activation signal. CD26 is also present on epithelial cells of various tissues, including the liver, kidney and intestine. Detailed analysis of subsets of human CD4+ lymphocytes indicates that CD26 appears to be more restricted than most other accessory molecules since it is expressed only on the CD4 memory/helper (CD45RO+CD29+) populations. This unique population of human CD4 cells is the only one that can respond to recall antigens, induce immunoglobulin G (IgG) synthesis and activate MHC-restricted cytotoxic T cells. In inflammatory diseases such as rheumatoid arthritis (RA), T cells at the sites of inflammation express the CD26 molecule strongly on the surface. See Morimoto C and Schlossman S F. Immunol Rev. 1998. 161:55-70.

Musashi (MSI) proteins are a family of RNA-binding proteins (RBPs) that are evolutionarily conserved across species. In mammals, two members of this family, Musashi1 (MSI1) and Musashi2 (MSI2), are strongly co-expressed in neural precursor cells, including CNS stem cells. MSI1 and MSI2 are RNA-binding proteins that are characterized by two RNP-type RNA recognition motifs (RRMs) and show remarkable similarity to one another, both in their primary structures and their RNA-binding specificities in vitro. In mammals, MSI1 and MSI2 expression is developmentally regulated. Both MSI1 and MSI2 are co-expressed predominantly in proliferating embryonic pluripotent neural precursors, as well as in cell populations that are believed to be the source of postnatal and adult CNS stem cells. In the cerebral cortex, the expression of MSI1 and MSI2 is rapidly down-regulated in newly generated postmitotic neurons, with the exception of some GABAergic interneurons that continue to express MSI2 exclusively. Although the molecular functions of the MSI family members remain obscure, their expression profiles suggest that they may play similar roles in the development and maintenance of CNS stem cells through posttranscriptional gene regulation. Mammalian MSI1 is expressed in fetal and adult NSCs and mature neurons. MSI2's CNS expression pattern is similar to MSI1 in terms of high expression levels in neural stem/progenitor cells, and MSI1 and MSI2 have been postulated to play mutually overlapping roles that remain to be elucidated. Nevertheless, MSI2 is continuously expressed in a subset of CNS neurons, particularly GABAergic neurons. Oligomeric assemblies of tau and the RNA-binding proteins (RBPs) Musashi (MSI) have been reported in Alzheimer's disease (AD). MSI1 protein was found to be present in tau inclusion-bearing neurons in AD and Pick's disease (PiD). Further, it has been shown that these two RBPs are present in their soluble aggregated, i.e., oligomeric forms in ex vivo human AD brains. These oligomers have been detected in mature neurons that can co-localize with oligomeric tau. See Sakakibara S et al. Dev. Biol. 1996. 176:230-242; Sakakibara S and Okano H. J Neurosci. 1997. 17:8300-8312; Sakakibara S et al. J Neurosci. 2001. 21:8091-8107; Keyoung H M et al. Nat. Biotechnol. 2001. 19:843-850; Okano H et al. J Cell Sci. 2002, 115:1355-1359; Sakakibara S et al. PNAS. 2002. 99(23): 15194-15199; Lovell M A, and Markesbery W R. J Neuropathol Exp Neurol. 2005. 64:675-680; Sengupta U et al. Acta Neuropathol Commun. 2018. 6(1): 113; Montalbano M et al. Nat Commun. 2020. 11(1): 4305.

Triggering receptor expressed on myeloid cells 2 (TREM2) belongs to the TREM family of cell surface transmembrane glycoproteins with V-immunoglobulin extra-cellular domains and cytoplasmic tails. The TREM2 gene is expressed in a subgroup of myeloid cells including dendritic cells, granulocytes, and tissue-specific macrophages like osteoclasts, Kuppfer cells and alveolar macrophages. In the brain, TREM2 is exclusively expressed by microglia. The expression of TREM2 varies depending on the particular region of the central nervous system (CNS), with a higher expression in the hippocampus, the spinal cord and the white matter. Expression of anti-inflammatory molecules has been shown to enhance TREM2 expression, while expression of pro-inflammatory molecules, such as TNFα, IL1β or lipopolysaccharide (LPS), has been shown to decrease TREM2 expression in vitro. TREM2 expression is up regulated in pathological conditions such as Parkinson's disease (PD), Amyotrophic lateral sclerosis (ALS), stroke, traumatic brain injury and AD. In AD, increased expression of TREM2 has been confirmed in patients and in mouse models of amyloid and tau pathology and seems to be associated with the recruitment of microglia to amyloid plaques. Further, aging-related increases TREM2 expression have been shown in both mice and humans. See Gratuze M et al. Mol Neurodegener. 2018. 13(1): 66; Carmona S et al. Lancet Neurol. 2018. 17(8): 721-730.

Interferon Regulatory Factor 4 (IRF4) is one of nine IRF family members. All IRF proteins share similar structure containing an N-terminal DBD and, except IRF1 and IRF2, carry a C-terminal IRF-associated domain (IAD) that is responsible interactions with other family members or other transcription factors including ETS factors and AP1 (activator protein 1) family members. IRF4 is a critical regulator of many aspects of B- and T-cell differentiation and cell metabolism. In DCs, Irf4 is highly expressed in the CD4⁺ subset and in a fraction pDCs. In line with this expression pattern, tissue-resident CD4⁺ DCs and nearly half the pDC population are absent from the spleen of Irf4^{-/-} mice. IRF-4 is a hemopoietic transcription factor critical for activation of microglia/macrophages and modulation of inflammatory responses. The effects of IRF4 signaling on inflammation are pleiotropic, and vary depending on immune cell types and the pathological microenvironment that is regulated by both pro- and anti-inflammatory cytokines. Mechanistically, IRF4 is a quintessential ‘context-dependent’ transcription factor that regulates distinct groups of inflammatory mediators in a differential manner depending on their activation in different cell types including phagocytes, T-cell subtypes, and neuronal cells. See Seillet C and Belz G T Advances in Immunology. 2013. V120: 185-210; Mamun A A and Liu F. Neurol Neurother. 2017. 2(1).

Granulocyte Macrophage-Colony Stimulating Factor (GM-CSF) is a hematological growth factor that regulates the production, migration, proliferation, differentiation and function of hematopoietic cells. It was first identified as being able to induce, in vitro, the proliferation and differentiation of bone marrow progenitors into granulocytes and macrophages. In response to inflammatory stimuli, GM-CSF is released by various cell types including T lymphocytes, macrophages, fibroblasts and endothelial cells. GM-CSF then activates and enhances the production and survival of neutrophils, eosinophils, and macrophages. Native GM-CSF is usually produced near the site of action where it modulates in vitro proliferation, differentiation, and survival of hematopoietic progenitor cells, but is present in circulating blood in only picomolar concentrations (10⁻¹⁰ to 10⁻¹² M). Several studies have shown that GM-CSF has a wide range of functions across different tissues in its action on myeloid cells, and GM-CSF deletion/depletion approaches have indicated its potential as an important therapeutic target in several inflammatory and autoimmune disorders. See A Metcalf D. Immunol Cell Biology. 1987, 65:35-43; Gasson J C. Blood. 1991, 77:1131-1145; Shannon M F et al. Crit Rev Immunol. 1997, 17:301-323; Alexander W S. Int Rev Immunol. 1998, 16:651-682; Barreda D R et al. Dev Comp Immunol. 2004, 28:509-554; Lee K M C et al. Immunotargets Ther. 2020. 9:225-240.

Recombinant human granulocyte-macrophage colony-stimulating factor (rhu GM-CSF) has been approved by the FDA for the treatment of neutropenia, blood dyscrasias and malignancies like leukemia in combination with chemotherapies. In the clinic, GM-CSF used for treatment of neutropenia and aplastic anemia following chemotherapy greatly reduces the risk of infection associated with bone marrow transplantation. Its utility in myeloid leukemia treatment and as a vaccine adjuvant is also well established. See Dorr R T. Clin Therapeutics. 1993. 15(1): 19-29; Armitage J O. Blood 1998, 92:4491-4508; Kovacic J C et al. J Mol Cell Cardiol. 2007, 42:19-33; Jacobs P P et al. Microbial Cell Factories 2010, 9:93.

The identification of specific biomarkers can be used for the diagnosis, prognosis, or theranosis of neurodegenerative or neurological diseases or disorders. They can also be used to identify neurodegenerative or neurological diseases or disorders and ailments that do not respond to monotherapy alone, and those that might benefit from combination therapies. Such combination therapies can potentially increase the percentage of patients who respond to treatments. Hence, there remains a need for new and more effective biomarkers and combination treatments of neurodegenerative or neurological diseases or disorders.

### SUMMARY

Accordingly, in an aspect, the present disclosure relates to a method for treating a neurodegenerative or neurological disease or disorder, comprising: administering an effective amount of a composition comprising GM-CSF to a patient in need thereof, wherein the patient is characterized by an increased or high expression and/or activity of Cluster of Differentiation 26 (CD26).

In an aspect, the present disclosure relates to a method for treating a neurodegenerative or neurological disease or disorder, comprising: administering an effective amount of a composition comprising GM-CSF to a patient in need thereof, wherein the patient is characterized by an increased or high expression and/or activity of one or more of MSI family proteins, MSI-1 or MSI-2.

In an aspect, the present disclosure relates to a method for treating a neurodegenerative or neurological disease or disorder, comprising: administering an effective amount of a composition comprising GM-CSF to a patient in need thereof, wherein the patient is characterized by an decreased or low expression and/or activity of TREM2.

In an aspect, the present disclosure relates to a method for treating a neurodegenerative or neurological disease or disorder, comprising: administering an effective amount of a composition comprising GM-CSF to a patient in need thereof, wherein the patient is characterized by an decreased or low expression and/or activity of IRF-4.

In an aspect, the present disclosure provides a method for treating a neurodegenerative or neurological disease or disorder, comprising: (a) identifying a patient undergoing or having undergone treatment with an agent for a neurodegenerative or neurological disease or disorder and presenting as failed, intolerant, resistant, or refractory to the treatment with an immunomodulatory or neurological agent; (b) determining the presence, absence or amount of CD26 and/or one or more MSI proteins and/or TREM2 and/or IRF-4 in a sample from the patient; and (c) administering an effective amount of a granulocyte-macrophage colony-stimulating factor (GM-CSF) agent to a patient demonstrating (i) an increased or high expression and/or activity of CD26 and/or MSI family proteins relative to a pre-treated and/or undiseased state and/or (ii) a decreased or low expression and/or activity of TREM2 and/or IRF4 to a pre-treated and/or undiseased state.

In an aspect, the present disclosure provides a method for selecting a patient for treatment with GM-CSF for a neurodegenerative or neurological disease or disorder based on the presence, absence or amount of CD26 and/or one or more MSI proteins and/or TREM2 and/or IRF-4 in a sample from the patient.

### BRIEF DESCRIPTION OF DRAWINGS

FIG. 1 illustrates a graph depicting the kinetics of expression of CD26 induced by sargramostim (LEUKINE). Monocytes and lymphocytes from multiple human donors (n=3) were treated with sargramostim (LEUKINE) at various concentrations 0.001 pM to 10 nM. Expression of CD26 was assessed on day 1 on both monocytes and lymphocytes. For reference, at 10 nM on X-axis, the upper curve is lymphocytes and the lower curve is monocytes.

FIG. 2 illustrates graphs depicting the kinetics of expression of Musashi-2 (MSI-2) induced by sargramostim (LEUKINE). Monocytes from a human donor were treated with sargramostim (LEUKINE) at various concentrations 1 pM to 10³ pM. Expression of Musashi-2 (MSI-2) was assessed on day 1 on monocytes (shown as the curve on the graph).

FIG. 3A illustrates a graph depicting the kinetics of expression of TREM2 induced by sargramostim (LEUKINE). Monocytes and lymphocytes from multiple human donors (n=3) were treated with sargramostim (LEUKINE) at various concentrations 0.001 nM to 10 nM. Expression of TREM2 was assessed on day 1 on both monocytes and lymphocytes. For reference, at 10 nM on X-axis, the upper curve is monocytes and the lower curve is lymphocytes.

FIG. 3B illustrates graphs depicting the kinetics of expression of TREM2 induced by sargramostim (LEUKINE). Monocytes from a human donor were treated with sargramostim (LEUKINE) at various concentrations 0.001 nM to 10 nM. Expression of TREM2 was assessed on day 1, day 2 and day 3 on monocytes. For reference, at 10 nM on X-axis, the top curve is the TREM2 expression on day 3, the middle curve is the TREM2 expression on day 2 and the bottom curve is the TREM2 expression on day 1.

FIG. 4 illustrates a graph depicting the kinetics of expression of IRF-4 induced by sargramostim (LEUKINE). Monocytes and lymphocytes from multiple human donors (n=3) were treated with sargramostim (LEUKINE) at various concentrations 0.03 nM to 100 nM. Expression of IRF-4 was assessed on day 1 on both monocytes and lymphocytes. For reference, at 10 nM on X-axis, the upper curve is monocytes and the lower curve is lymphocytes.

### DETAILED DESCRIPTION

The present disclosure relates to, in part, to the use of GM-CSF as an effective treatment for specific neurodegenerative or neurological diseases or disorders, selected using CD26 and/or one or more MSI proteins and/or TREM2 and/or IRF-4 as a predictive marker of disease sequelae and responsiveness to current therapy, e.g. with an agent to treat a neurodegenerative or neurological disease or disorder.

In aspects, the present disclosure relates to improved treatments for neurodegenerative or neurological diseases or disorders in a patient that has failed, is intolerant, is resistant, or is refractory to the current treatment in use. For instance, in embodiments, evaluation of the presence, absence, levels or activity of CD26, one or more MSI proteins, TREM2 and/or IRF-4 informs or predicts the disease state in the and, without limitation, the administration of GM-CSF converts the patient that has failed, is intolerant, is resistant, or is refractory to current treatment(s) for the neurological indication to a patient who responds to current treatment(s) for the neurological indication. In embodiments, the GM-CSF modulates CD26, or cells expressing the same, to improve a patient's treatment outcome. In embodiments, the GM-CSF modulates one or more MSI proteins, e.g. MSI-1 or MSI-2, or cells expressing the same, to improve a patient's treatment outcome. In embodiments, the GM-CSF modulates TREM-2, or cells expressing the same, to improve a patient's treatment outcome. In embodiments, the GM-CSF modulates IRF-4, or cells expressing the same, to improve a patient's treatment outcome.

Accordingly, in aspects, the disclosure provides methods for treating neurodegenerative or neurological diseases or disorders. In embodiments, the neurodegenerative or neurological disease or disorder is selected from Alzheimer's disease, Parkinson's disease, progressive supranuclear palsy (PSP), multiple system atrophy (MSA), Lewy body dementia, Parkinson's disease dementia epilepsy, stroke, Huntington's Chorea or Huntington's Disease (HD), cerebral hypoxia, multiple sclerosis, amyotrophic lateral sclerosis (ALS), neovascular glaucoma, optic neuropathy, spinal muscular atrophy (SMA), spinocerebellar ataxia (SCA), and peripheral neuropathy.

Compositions of Immunomodulatory and/or Neurological Agents

In embodiments, the present disclosure pertains to pharmaceutical compositions comprising the compositions, e.g. GM-CSF and/or an additional therapeutics to treat a neurodegenerative or neurological disease or disorder.

In embodiments, the additional immunomodulatory or neurological agent is selected from dopamine precursors such as Levodopa, cholinesterase inhibitors such as donepezil (ARICEPT), rivastigmine (EXELON), Galantamine (RAZADYNE), atypical antipsychotics/second generation antipsychotics including serotonin-dopamin antagonists (SDAs), multi-acting receptor-targeted antipsychotics (MARTAs), and D2 partial agonists (e.g. ABILIFY/Aripiprazol), NMDA receptor antagonist memantine, riluzole (RILUTEK), NSAIDs (non-steroidal anti-inflammatory drugs), caffein A2A receptor antagonists and CERE-120 (adeno-associated virus serotype 2-neurturin), deep brain stimulation, TNF-α antagonists including etanercept, adalimumab, infliximab, IFN-γ inhibitors, TGF-β modulators, IL-33 inhibitors, IL-18 inhibitors, VEGF inhibitors, IL-1 inhibitors, inhibitors of pathological beta-amyloid (Aβ) plaques, such as Aβ-directed monoclonal antibodies such as aducanumab (ADUHELM), NSAIDs such as metacetamol and asprin, anti-diabetic drugs such as linagliptin, suppressors of tau-activation such as liraglutide, miRNA's that target Aβ-plaque formation and tau protein phosphorylation, α-secretase enhancers such as Ginkgo biloba and salvia miltiorrhiza, β-secretase inhibitors such as huanglian and yuanzhi.

In embodiments, the immunoregulatory or neurological agent is an antibody or antibody format which is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

### Compositions of GM-CSF

GM-CSF, in embodiments, includes any pharmaceutically safe and effective GM-CSF, or any derivative thereof having the biological activity of GM-CSF. In embodiments, the GM-CSF is rhu GM-CSF, such as sargramostim (LEUKINE). Sargramostim is a biosynthetic, yeast-derived, recombinant human GM-CSF, having a single 127 amino acid glycoprotein that differs from endogenous human GM-CSF by having a leucine instead of a proline at position 23. Other natural and synthetic GM-CSFs, and derivatives thereof having the biological activity of natural human GM-CSF, may be equally useful in embodiments.

In embodiments, the GM-CSF is produced or producible in bacteria, yeasts, plants, insect cells, and mammalian cells. In embodiments, the GM-CSF is produced or producible in Escherichia coli cells. In embodiments, the GM-CSF is produced or producible in yeast cells. In embodiments, the GM-CSF is produced or producible in Chinese hamster ovary cells (CHO). In embodiments, the GM-CSF is not produced in E. coli cells. In embodiments, the GM-CSF is produced in a cell that allows for glycosylation, e.g. yeast or CHO cells.

In embodiments, the GM-CSF has an amino acid sequence of SEQ ID NO: 1, or a variant of at least about 90%, or at least about 93%, or at least about 95%, or at least about 97%, or at least about 98% identity thereto. In embodiments, the GM-CSF has an amino acid sequence of SEQ ID NO: 2, SEQ ID NO: 3, and SEQ ID NO:4, or a variant of at least about 90%, or at least about 93%, or at least about 95%, or at least about 97%, or at least about 98% identity thereto. In embodiments, the GM-CSF is one of, sargramostim, molgramostim, and regramostim. In embodiments, the GM-CSF is sargramostim,

Without wishing to be bound by theory, the core of hGM-CSF consists of four helices that pack at angles. Crystal structures and mutagenic analysis of rhGM-CSF (Rozwarski D A et al., Proteins 26:304-13, 1996) showed that, in addition to apolar side chains in the protein core, 10 buried hydrogen bonding residues involve intramolecular hydrogen bonding to main chain atoms that were better conserved than residues hydrogen bonding to other side chain atoms; 24 solvation sites were observed at equivalent positions in the two molecules in the asymmetric unit, and the strongest among these was located in clefts between secondary structural elements. Two surface clusters of hydrophobic side chains are located near the expected receptor binding regions. Mutagenesis of residues on the helix A/helix C face confirmed the importance of certain Glu, Gly, and Gln residues. These residues are therefore not to be substituted in the functional substitution variants of hGM-CSF for use in the present disclosure and these helices are to be retained in a functional fragments or deletion variants of hGM-CSF for use in this disclosure. Further, in embodiments, one of ordinary skill can reference UniProtKB entry P04141 for structure information to inform the identity of variants.

The N-terminal helix of hGM-CSF governs high affinity binding to its receptor (Shanafelt A B et al., EMBO J 10:4105-12, 1991) Transduction of the biological effects of GM-CSF requires interaction with at least two cell surface receptor components, (one of which is shared with the cytokine IL-5). The above study identified receptor binding determinants in GM-CSF by locating unique receptor binding domains on a series of human-mouse hybrid GM-CSF cytokines. The interaction of GM-CSF with the shared subunit of their high affinity receptor complexes was governed by a very small part of the peptide chains. The presence of a few key residues in the N-terminal a-helix of was sufficient to confer specificity to the interaction.

In embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt a-helices.

As used herein, “non-conservative substitutions” are defined as exchanges of an amino acid by another amino acid listed in a different group of the six standard amino acid groups (1) to (6) shown above.

In embodiments, the substitutions may also include non-classical amino acids (e.g. selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

Modification of the amino acid sequences may be achieved using any known technique in the art e.g., site-directed mutagenesis or PCR based mutagenesis. Such techniques are described, for example, in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N.Y., 1989. Without wishing to be bound by theory, the degree of glycosylation of biosynthetic GM-CSFs appears to influence half-life, distribution, and elimination. (Lieschke and Burgess, N. Engl. J. Med. 327:28-35, 1992; Dorr, R. T., Clin. Ther. 15:19-29, 1993; Horgaard et al., Eur. J. Hematol. 50:32-36, 1993). In embodiments, the present GM-CSF molecules are glycosylated.

### Biomarker

In aspects, the present methods relate to the utility of a predictive biomarkers to determine the use of GM-CSF in the treatment of neurodegenerative or neurological diseases or disorders.

In one aspect, the present disclosure relates to a method of treating a patient in need of therapy wherein the patient is characterized by having failed or being intolerant or refractory to a treatment with an immunomodulatory or neurological agent. In other aspects, an assessment of the patient having failed or being intolerant or refractory to a treatment with an immunomodulatory or neurological agent comprises measuring of a biomarker in a sample of the patient.

In embodiments, an assessment of the patient having failed or being intolerant or refractory to a treatment with an immunomodulatory or neurological agent comprises measuring a variety of patient parameters. In embodiments, the patient sample may be analyzed using, e.g. immunohistochemical or immunofluorescence techniques may be used to evaluate the immune infiltrate, for example, immune subsets such as, CD4⁺ Th cells (T helper cells), IL-17-producing CD4⁺ Th cells (Th17 cells), CD8⁺ T cells (cytotoxic T cells), and systemic or circulating intermediate monocytes. In embodiments, polychromatic flow cytometry can be used to measure multiple surface and intracellular markers, allowing characterization of cell phenotype and activation state. In embodiments, whole blood can be used to evaluate changes in cell count with therapy or changes in cytokine levels, for example IL-1, IL-4, IL-6, IL-10, IL-12, IL-18, IL-33, IFN-γ, IP-10, M-CSF, TGF-β, VEGF, and TNFα. In embodiments, deep sequencing techniques can be used to yield quantification of changes in individual cell clonotypes.

In embodiments, an assessment of the patient having failed or being intolerant or refractory to a treatment with an immunomodulatory or neurological agent comprises measuring the presence, absence, or amount of Cluster of Differentiation 26 (CD26) isotype in a sample of the patient.

In embodiments, an assessment of the patient having failed or being intolerant or refractory to a treatment with an immunomodulatory or neurological agent comprises measuring the presence, absence, or amount of one or more MSI family proteins isotype in a sample of the patient.

In embodiments, an assessment of the patient having failed or being intolerant or refractory to a treatment with an immunomodulatory or neurological agent comprises measuring the presence, absence, or amount of TREM2 isotype in a sample of the patient.

In embodiments, an assessment of the patient having failed or being intolerant or refractory to a treatment with an immunomodulatory or neurological agent comprises measuring the presence, absence, or amount of IRF-4 isotype in a sample of the patient.

In embodiments, the present disclosure relates to a method for treating a neurodegenerative or neurological disease or disorder, wherein CD26 is used as a biomarker for predicting or determining the need for treatment with GM-CSF.

In embodiments, the present disclosure relates to a method for treating a neurodegenerative or neurological disease or disorder, wherein one or more MSI family proteins is used as a biomarker for predicting or determining the need for treatment with GM-CSF.

In embodiments, the present disclosure relates to a method for treating a neurodegenerative or neurological disease or disorder wherein TREM2 is used as a biomarker for predicting or determining the need for treatment with GM-CSF.

In embodiments, the present disclosure relates to a method for treating a neurodegenerative or neurological disease or disorder wherein IRF-4 is used as a biomarker for predicting or determining the need for treatment with GM-CSF.

In embodiments, the presence, absence or amount of CD26, one of more MSI proteins, TREM2 and/or IRF-4 by detection of protein and/or nucleic acids in a sample of the patient.

In embodiments, the presence, absence or amount of CD26, one of more MSI proteins, TREM2 and/or IRF-4 is determined by ELISA, immunohistochemical staining, western blotting, in-cell western, immunofluorescent staining, or fluorescent activating cell sorting (FACS), or the like, in a sample of the patient.

In embodiments, the method for determining the presence, absence or amount of CD26, one or more MSI proteins, TREM2 and/or IRF-4 is a method of characterizing a patient or selecting a patient for the treatment comprising GM-CSF.

In embodiments, the method of determining the levels of CD26, MSI family proteins, TREM2 and/or IRF4, involves assaying the levels of CD26, MSI family proteins, TREM2 and/or IRF4 in a biological sample from the patient.

In embodiments, the present methods, e.g., the method of determining CD26, one or more MSI proteins, TREM2 and/or IRF-4 presence, absence, levels or activity for the purposes of patient selection, employs a sample, the sample selected from blood, skin sample or tissue sample, plasma, serum, pus, urine, perspiration, tears, mucus, sputum, saliva, cerebrospinal fluid (CSF) and/or other body fluids.

In embodiments, the method of patient selection is undertaken using a sample of the patient, where the sample is selected from blood, skin sample or tissue sample, tissue biopsy, a formalin-fixed or paraffin-embedded tissue specimen, cytological sample, cultured cells, plasma, serum, pus, urine, perspiration, tears, mucus, sputum, saliva, cerebrospinal fluid (CSF) and/or other body fluids.

In embodiments, the present methods direct patient treatment decisions. For instance, in embodiments, the method comprises the step of monitoring the expression and/or activity of CD26 and/or one or more MSI proteins during the course of treatment. In embodiments, the methods may detect a high or increased CD26 and/or one or more MSI proteins expression or activity, and this is correlative with the patient having failed or being intolerant or refractory to a treatment with an immunomodulatory or neurological agent. In such embodiments, without limitation, this directs treatment of the patient with GM-CSF agents. In embodiments, the patient with an increased expression and/or activity of CD26 and/or one or more MSI proteins directs continued administration of GM-CSF. In embodiments, the patient with an increased or high expression or activity of CD26 and/or one or more MSI proteins receives, for example, a greater dose of GM-CSF, and/or additional neurological therapies. In embodiments, the patient with a decreased expression and/or activity of CD26 and/or one or more MSI proteins directs discontinuation of administration of GM-CSF.

In embodiments, the present method comprises the step of monitoring the expression and/or activity of TREM2 and/or IRF-4 during the course of treatment. In embodiments, the methods may detect a low or decreased TREM2 and/or IRF-4 expression or activity, and this is correlative with the patient having failed or being intolerant or refractory to a treatment with an immunomodulatory or neurological agent. In such embodiments, without limitation, this directs treatment of the patient with GM-CSF agents. In embodiments, the patient with an decreased expression and/or activity of TREM2 and/or IRF-4 directs continued administration of GM-CSF. In embodiments, the patient with an decreased or low expression or activity of TREM2 and/or IRF-4 receives, for example, a greater dose of GM-CSF, and/or additional neurological therapies. In embodiments, the patient with an increased expression and/or activity of TREM2 and/or IRF-4 directs discontinuation of administration of GM-CSF.

In embodiments, the GM-CSF agents, as described herein, potentiate treatment with an immunomodulatory or neurological agent. In embodiments, GM-CSF agents, as described herein, are used to modulate the patient's immune system, e.g. by decreasing or increasing expression and/or activity of CD26, one or more MSI proteins, TREM2 and/or IRF-4.

### Methods of Treatment

In one aspect, the present disclosure relates to a method for treating a neurodegenerative or neurological disease or disorder, comprising: administering an effective amount of a composition GM-CSF to a patient in need thereof.

In another aspect, the present disclosure relates to a method for treating a neurodegenerative or neurological disease or disorder, comprising: administering an effective amount of a composition comprising GM-CSF in conjunction with an immunomodulatory or neurological agent to a patient in need thereof, wherein the patient is characterized by the presence, absence or amount of CD26, one or more MSI proteins, TREM2 and/or IRF-4 in a sample of the patient.

In aspects, the present disclosure relates to methods for treating a neurodegenerative or neurological disease or disorder, comprising: (a) identifying a patient undergoing or having undergone treatment with an agent for neurological issues and presenting as failed, intolerant, resistant, or refractory to the treatment with an immunomodulatory or neurological agent; (b) determining the presence, absence or amount of CD26, one or more MSI proteins, TREM2 and/or IRF-4 in a sample from the patient; and (c) (i) administering an effective amount of a granulocyte-macrophage colony-stimulating factor (GM-CSF) agent to a patient demonstrating an increased or high expression and/or activity of CD26 and/or one or more MSI proteins relative to a pre-treated and/or undiseased state, or (c) (ii) administering an effective amount of a granulocyte-macrophage colony-stimulating factor (GM-CSF) agent to a patient demonstrating an decreased or low expression and/or activity of TREM2 and/or IRF-4 relative to a pre-treated and/or undiseased state.

In aspects, the present disclosure relates to methods of treating a neurodegenerative or neurological disease or disorder, comprising: (a) selecting a patient having neurodegenerative or neurological disease or disorder and one or more of (i) increased expression and/or activity of CD26 relative to a non-diseased state; (ii) increased expression and/or activity of one or more MSI proteins relative to a non-diseased state; (iii) decreased expression and/or activity of TREM2 relative to a non-diseased state; (iv) decreased expression and/or activity of IRF-4 relative to a non-diseased state and (b) administering an effective amount of a composition comprising GM-CSF to the patient.

In embodiments, the present disclosure relates to a method for treating a neurodegenerative or neurological disease or disorder, comprising: administering an effective amount of a composition comprising GM-CSF alone or in conjunction with an immunomodulatory or neurological agent to a patient in need thereof, wherein the patient is characterized by as a partial responder or a non-responder to a neurological treatment.

In embodiments, the present disclosure relates to a method for treating cancer, comprising: administering an effective amount of a composition comprising GM-CSF alone or in conjunction with an immunomodulatory or neurological agent to a patient in need thereof, wherein the patient is characterized by having failed or being intolerant or refractory to a treatment with an immunomodulatory or neurological agent.

In embodiments, the method of treatment causes a decrease in the expression and/or activity of CD26. In embodiments, the method of treatment causes a decrease in the expression and/or activity of one or more MSI proteins.

In embodiments, the method of treatment causes an increase in the expression and/or activity of TREM2. In embodiments, the method of treatment causes an increase in the expression and/or activity of IRF-4.

In embodiments, the method of treatment prevents, treats, and/or mitigates progression and/or development of the neurodegenerative or neurological disease or disorder in the patient. In embodiments, the method of treatment improves the neurodegenerative or neurological disease or disorder in the patient. In embodiments, the method of treatment elicits a disease-modifying response in the patient. In other embodiments, the method of treatment elicits temporarily or permanently slows down cognitive decline in the patient. In still other embodiments, the method of treatment causes an amelioration of the neurodegenerative or neurological disease or disorder symptoms. In yet other embodiments, the method of treatment slows the onset and/or development of the neurodegenerative or neurological diseases or disorders.

In embodiments, the method of treatment decreases or mitigates reverses or prevents chronic inflammation in the central nervous system (CNS). In embodiments, the method of treatment decreases or mitigates the dysfunction of endogenous or exogenous CNS immune cells. In embodiments, the method decreases or mitigates the activation of CNS astrocytes and mononuclear phagocytes, for example perivascular macrophages and microglial cells.

In embodiments, the method of treatment decreases or mitigates or reverses astrogliopathy. In embodiments, the method of treatment modulates the expression of one or more cytokines and/or proteins.

In embodiments, the method of treatment modulates or maintains or supports the glutamine-glutamate balance in the CNS. In embodiments, the method of treatment decreases or mitigates or reverses chronic microglial cell activation. In embodiments, the method of treatment decreases or reverses axonal damage.

In embodiments the method of treatment decreases or prevents amyloid pathologies. In embodiments, the method of treatment causes a decrease or prevents taupathy.

In embodiments, the method of treatment causes a decrease in the sequelae of a neurodegenerative or neurological disease or disorder in the patient relative to before treatment.

In embodiments, the method of treatment reverses or prevents excessive production and/or signaling of one or more inflammatory cytokines, such as IL-1, IL-4, IL-6, IL-10, IL-12, IL-18, IL-33, IFN-g, IP-10, M-CSF, TGF-b, VEGF, and TNFα.

In embodiments, the method of treatment decreases or prevents amyloid pathologies. In embodiments, the method of treatment causes a decrease or prevents taupathy.

In embodiments, the agent that stimulates the survival, proliferation and activation of neutrophils, macrophages and/or dendritic cells is administered at a time selected from (i) the same time as an immunomodulatory or neurological agent; (ii) within about 1 hour, about 2 hours, about 4 hours, about 8 hours, about 12 hours, about 24 hours, about 36 hours, about 48 hours, about 72 hours, or about 96 hours or about 1 week or about 2 weeks following administration of said neurological agent; (iii) at least about 1 hour, about 2 hours, about 4 hours, about 8 hours, about 12 hours, about 24 hours, about 48 hours, about 36 hours, about 72 hours, or about 96 hours, or about 1 week or about 2 weeks prior to administration of the neurological agent; and/or (iv) after at least an about 10%, about 20%, about 30%, about 40% or about 50% decrease in expression of an extracellular marker such as CD86, CD109 and/or CD122.

In embodiments, the neurodegenerative or neurological disease or disorder is selected from one or more of Alzheimer's disease, Parkinson's disease, progressive supranuclear palsy (PSP), multiple system atrophy (MSA), Lewy body dementia, Parkinson's disease dementia epilepsy, stroke, Huntington's Chorea or Huntington's Disease (HD), cerebral hypoxia, multiple sclerosis, amyotrophic lateral sclerosis (ALS), neovascular glaucoma, optic neuropathy, spinal muscular atrophy (SMA), spinocerebellar ataxia (SCA), and peripheral neuropathy. In embodiments, the patient is afflicted with Alzheimer's disease or Parkinson's disease.

In embodiments, the patient is afflicted with a chronic, progressive disorder of the nervous system.

In embodiments, the patient is characterized by having oxidative stress, loss of neurite integrity, apoptosis, neuronal loss or/and inflammation response, cognitive impairment, cognitive decline, behavioral and personality changes, tremors, bradykinesia, rigidity, impaired posture and balance, loss of automatic movements, decrease in motor coordination, changes in speech, photophobia, difficulty controlling eye muscles, slowed saccadic eye movements, dysphagia, blepharospasm, fainting or lightheadedness due to orthostatic hypotension, dizziness, bladder control problems, well-formed visual hallucinations and delusions, changes in memory, concentration and judgement, memory loss, depression, irritability, anxiety, rapid eye movement (REM) sleep disorder, epileptic seizures, dysesthesia, numbness or tingling, spasticity, difficulty chewing or swallowing, muscle twitching and weakness in a limb, and/or prickling or tingling in feet or hands.

In embodiments, the method further comprises administering one or more additional therapeutic agents, selected from dopamine precursors such as Levodopa, cholinesterase inhibitors such as donepezil (ARICEPT), rivastigmine (EXELON), Galantamine (RAZADYNE), atypical antipsychotics/second generation antipsychotics including serotonin-dopamin antagonists (SDAs), multi-acting receptor-targeted antipsychotics (MARTAs), and D2 partial agonists (e.g. ABILIFY/Aripiprazol), NMDA receptor antagonist memantine, riluzole (RILUTEK), NSAIDs (non-steroidal anti-inflammatory drugs), caffein A2A receptor antagonists and CERE-120 (adeno-associated virus serotype 2-neurturin), deep brain stimulation, TNF-α antagonists including etanercept, adalimumab, infliximab, IFN-γ inhibitors, TGF-β modulators, IL-33 inhibitors, IL-18 inhibitors, VEGF inhibitors, IL-1 inhibitors, inhibitors of pathological beta-amyloid (Aβ) plaques, such as Aβ-directed monoclonal antibodies such as aducanumab (ADUHELM), NSAIDs such as metacetamol and asprin, anti-diabetic drugs such as linagliptin, suppressors of tau-activation such as liraglutide, miRNA's that target Aβ-plaque formation and tau protein phosphorylation, α-secretase enhancers such as Ginkgo biloba and salvia miltiorrhiza, β-secretase inhibitors such as huanglian and yuanzhi.

### Pharmaceutically Acceptable Salts and Excipients

The compositions described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

Pharmaceutically acceptable salts include, by way of non-limiting example, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts.

The term “pharmaceutically acceptable salt” also refers to a salt of the compositions of the present disclosure having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

In embodiments, the compositions described herein are in the form of a pharmaceutically acceptable salt.

### Pharmaceutical Compositions and Formulations

In embodiments, the present disclosure pertains to pharmaceutical compositions comprising the compositions, e.g. GM-CSF and/or an additional therapeutic agent, e.g. the therapeutic agent described herein, and a pharmaceutically acceptable carrier or excipient.

In embodiments, the additional therapeutic agent comprises and/or is selected from dopamine precursors such as Levodopa, cholinesterase inhibitors such as donepezil (ARICEPT), rivastigmine (EXELON), Galantamine (RAZADYNE), atypical antipsychotics/second generation antipsychotics including serotonin-dopamin antagonists (SDAS), multi-acting receptor-targeted antipsychotics (MARTAS), and D2 partial agonists (e.g. ABILIFY/Aripiprazol), NMDA receptor antagonist memantine, riluzole (RILUTEK), NSAIDs (non-steroidal anti-inflammatory drugs), caffein A2A receptor antagonists and CERE-120 (adeno-associated virus serotype 2-neurturin), deep brain stimulation, TNF-α antagonists including etanercept, adalimumab, infliximab, IFN-γ inhibitors, TGF-β modulators, IL-33 inhibitors, IL-18 inhibitors, VEGF inhibitors, IL-1 inhibitors, inhibitors of pathological beta-amyloid (Aβ) plaques, such as Aβ-directed monoclonal antibodies such as aducanumab (ADUHELM), NSAIDs such as metacetamol and asprin, anti-diabetic drugs such as linagliptin, suppressors of tau-activation such as liraglutide, miRNA's that target Aβ-plaque formation and tau protein phosphorylation, α-secretase enhancers such as Ginkgo biloba and salvia miltiorrhiza, β-secretase inhibitors such as huanglian and yuanzhi, and pharmaceutically acceptable salts, acids or derivatives of any of the above.

Any pharmaceutical compositions described herein can be administered to a patient as a component of a composition that comprises a pharmaceutically acceptable carrier or vehicle. Such compositions can optionally comprise a suitable amount of a pharmaceutically acceptable excipient so as to provide the form for proper administration.

In embodiments, pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a patient. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

The present disclosure includes the described pharmaceutical compositions (and/or additional therapeutic agents) in various formulations. Any inventive pharmaceutical composition (and/or additional therapeutic agents) described herein can take the form of solutions, suspensions, emulsion, drops, tablets, pills, pellets, capsules, capsules containing liquids, gelatin capsules, powders, sustained-release formulations, suppositories, emulsions, aerosols, sprays, suspensions, lyophilized powder, frozen suspension, desiccated powder, or any other form suitable for use. In one embodiment, the composition is in the form of a capsule. In another embodiment, the composition is in the form of a tablet. In yet another embodiment, the pharmaceutical composition is formulated in the form of a soft-gel capsule. In embodiments, the pharmaceutical composition is formulated in the form of a gelatin capsule. In yet another embodiment, the pharmaceutical composition is formulated as a liquid.

Where necessary, the inventive pharmaceutical compositions (and/or additional therapeutic agents) can also include a solubilizing agent. Also, the agents can be delivered with a suitable vehicle or delivery device as known in the art. Combination therapies outlined herein can be co-delivered in a single delivery vehicle or delivery device.

The formulations comprising the inventive pharmaceutical compositions (and/or additional therapeutic agents) of the present disclosure may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In embodiments, any pharmaceutical compositions (and/or additional therapeutic agents) described herein is formulated in accordance with routine procedures as a composition adapted for a mode of administration described herein.

Routes of administration include, for example: topical, oral, intradermal, transdermal, subcutaneous, intramuscular, intraperitoneal, intravenous, intranasal, epidural, sublingual, intranasal, intracerebral, intravaginal, rectal, or by inhalation. Administration can be local or systemic. In embodiments, the administering is by an intravenous route. The mode of administration can be left to the discretion of the practitioner, and depends in-part upon the site of the medical condition. In most instances, administration results in the release of any agent described herein onto or into the affected site.

In specific embodiments, the GM-CSF (and/or additional therapeutic agents) is administered via an intravenous route.

In one embodiment, the pharmaceutical compositions (and/or additional therapeutic agents) described herein are formulated in accordance with routine procedures as a composition adapted for administration. For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Dosage forms suitable for parenteral administration (e.g. intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents known in the art. Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

Compositions for oral delivery can be in the form of tablets, lozenges, aqueous or oily suspensions, granules, powders, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation.

Compositions for topical delivery can be in the form of a cream, gel, ointment, lotion, spray, aqueous or oily suspensions, powders, or emulsions, for example. Increased skin permeability and penetration may be achieved by non-invasive methods, for example, with the use of any nanocarriers combined with any pharmaceutical composition (and/or additional therapeutic agents) described herein. The skin can act as a reservoir, and can be used to deliver the compositions (and/or additional therapeutic agents) described herein for more extended periods in a sustained manner.

Any inventive pharmaceutical compositions (and/or additional therapeutic agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropyl cellulose, hydropropylmethyl cellulose, polyvinylpyrrolidone, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the agents described herein. The disclosure thus provides single unit dosage forms suitable for oral administration such as, but not limited to, tablets, capsules, gelcaps, and caplets that are adapted for controlled- or sustained-release.

Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

### Administration and Dosage

It will be appreciated that the actual dose of the composition to be administered according to the present disclosure will vary according to the particular dosage form, and the mode of administration. Many factors that may modify the action of the composition (e.g., body weight, gender, diet, time of administration, route of administration, rate of excretion, condition of the patient, drug combinations, genetic disposition and reaction sensitivities) can be taken into account by those skilled in the art. Administration can be carried out continuously or in one or more discrete doses within the maximum tolerated dose. Optimal administration rates for a given set of conditions can be ascertained by those skilled in the art using conventional dosage administration tests.

In embodiments, the GM-CSF is administered at a total dose of about 125 μg, about 150 μg, or about 200 μg, or about 250 μg, or about 300 μg, or about 350 μg. In embodiments, the GM-CSF is administered at a total dose of about 250 μg.

In embodiments, the GM-CSF is administered at a dose of about 125 μg, about 150 μg, or about 200 μg, or about 250 μg, or about 300 μg, or about 350 μg.

In embodiments, the GM-CSF is administered at a dosing schedule of once monthly, or twice monthly, or once weekly, or twice weekly, or once daily or twice daily. In embodiments, the GM-CSF is administrated weekly.

In embodiments, the GM-CSF is sargramostim, administered at a dose of about 125 μg, once weekly.

### Combination Therapy and Additional Therapeutic Agents

In embodiments, the pharmaceutical composition of the present disclosure is co-administered in conjunction with additional agent(s), for example an immunomodulatory or neurological agent, such as a checkpoint inhibitor. Co-administration can be simultaneous or sequential.

In one embodiment, the additional immunomodulatory or neurological agent and the GM-CSF of the present disclosure are administered to a patient simultaneously. The term “simultaneously” as used herein, means that the immunomodulatory or neurological agent and the GM-CSF are administered with a time separation of no more than about 60 minutes, such as no more than about 30 minutes, no more than about 20 minutes, no more than about 10 minutes, no more than about 5 minutes, or no more than about 1 minute. Administration of the immunomodulatory or neurological agent and the GM-CSF can be by simultaneous administration of a single formulation (e.g., a formulation comprising the additional therapeutic agent and the GM-CSF composition) or of separate formulations (e.g., a first formulation including the immunomodulatory or neurological agent and a second formulation including the GM-CSF composition).

Co-administration does not require the therapeutic agents to be administered simultaneously, if the timing of their administration is such that the pharmacological activities of the immunomodulatory or neurological agent and the GM-CSF overlap in time, thereby exerting a combined therapeutic effect. For example, the immunomodulatory or neurological agent and the targeting moiety, the GM-CSF composition can be administered sequentially. The term “sequentially” as used herein means that the immunomodulatory or neurological agent and the GM-CSF are administered with a time separation of more than about 60 minutes. For example, the time between the sequential administration of the immunomodulatory or neurological agent and the GM-CSF can be more than about 60 minutes, more than about 2 hours, more than about 5 hours, more than about 10 hours, more than about 1 day, more than about 2 days, more than about 3 days, more than about 1 week apart, more than about 2 weeks apart, or more than about one month apart. The optimal administration times will depend on the rates of metabolism, excretion, and/or the pharmacodynamic activity of the additional therapeutic agent and the GM-CSF being administered. Either the immunomodulatory or neurological agent or the GM-CSF composition may be administered first.

Co-administration also does not require the therapeutic agents to be administered to the patient by the same route of administration. Rather, each therapeutic agent can be administered by any appropriate route, for example, parenterally or non-parenterally.

In embodiments, the GM-CSF described herein acts synergistically when co-administered with the immunomodulatory or neurological agent. In such embodiments, the targeting moiety, the GM-CSF composition and the immunomodulatory or neurological agent may be administered at doses that are lower than the doses employed when the agents are used in the context of monotherapy.

### Samples

In embodiments, the sample is selected from a biopsy, a tissue and/or a body fluid.

In embodiments, the sample is selected from blood, skin sample or tissue sample, tissue biopsy, a formalin-fixed or paraffin-embedded tissue specimen, cytological sample, cultured cells, plasma, serum, pus, urine, perspiration, tears, mucus, sputum, saliva and/or other body fluids.

Sequences

SEQ ID NO: 1 is wild type GM-CSF:
APARSPSPSTQPWEHVNAIQEARRLLNLSRDTAAEMNETVEVISEMFDLQEPTCLQTRLELYKQ
GLRGSLTKLKGPLTMMASHYKQHCPPTPETSCATQIITFESFKENLKDFLLVIPFDCWEPVQE.
SEQ ID NO: 2 is sargramostim:
APARSPSPSTQPWEHVNAIQEALRLLNLSRDTAAEMNETVEVISEMFDLQEPTCLQTRLELYKQ
GLRGSLTKLKGPLTMMASHYKQHCPPTPETSCATQIITFESFKENLKDFLLVIPFDCWEPVQE.
SEQ ID NO: 3 is molgramostim:
APARSPSPSTQPWEHVNAIQEARRLLNLSRDTAAEMNETVEVISEMFDLQEPTCLQTRLELYKQ
GLRGSLTKLKGPLTMMASHYKQHCPPTPETSCATQIITFESFKENLKDFLLVIPFDCWEPVQE
SEQ ID NO: 4 is regramostim:
APARSPSPSTQPWEHVNAIQEARRLLNLSRDTAAEMNETVEVISEMFDLQEPTCLQTRLELYKQ
GLRGSLTKLKGPLTMMASHYKQHCPPTPETSCATQTTFESFKENLKDFLLVIPFDCWEPVQE

### Definitions

The following definitions are used in connection with the invention disclosed herein. Unless defined otherwise, all technical and scientific terms used herein have the same meaning as commonly understood to one of skill in the art to which this invention belongs.

An “effective amount,” when used in connection with an agent effective for the treatment of a coronavirus infection is an amount that is effective for treating or mitigating a coronavirus infection.

As used herein, “a,” “an,” or “the” can mean one or more than one. Further, the term “about” when used in connection with a referenced numeric indication means the referenced numeric indication plus or minus up to 10% of that referenced numeric indication. For example, the language “about 50” covers the range of 45 to 55.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

### EXAMPLES

### Example 1: Expression and Kinetics of CD26, MSI proteins, TREM2 and IRF-4 on Human Monocytes

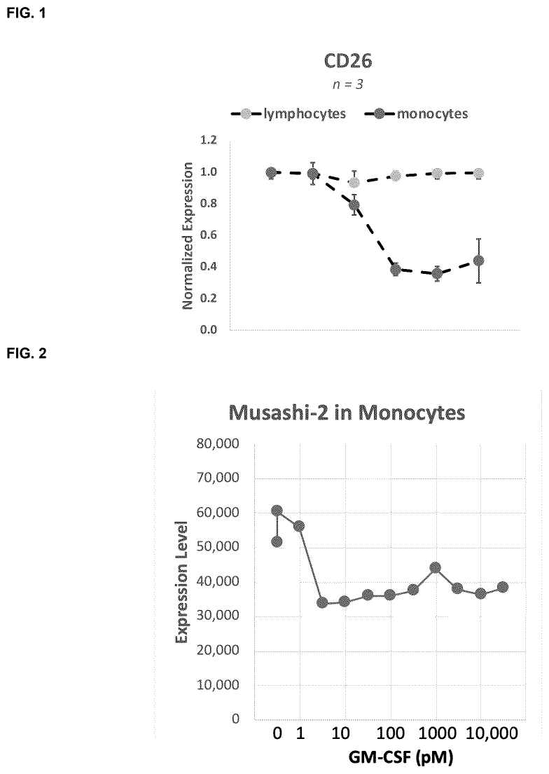

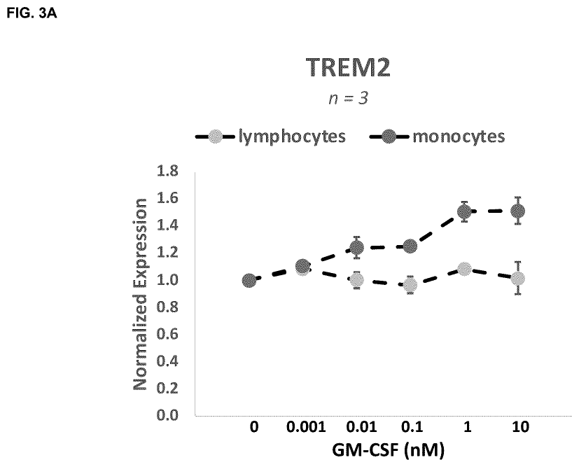

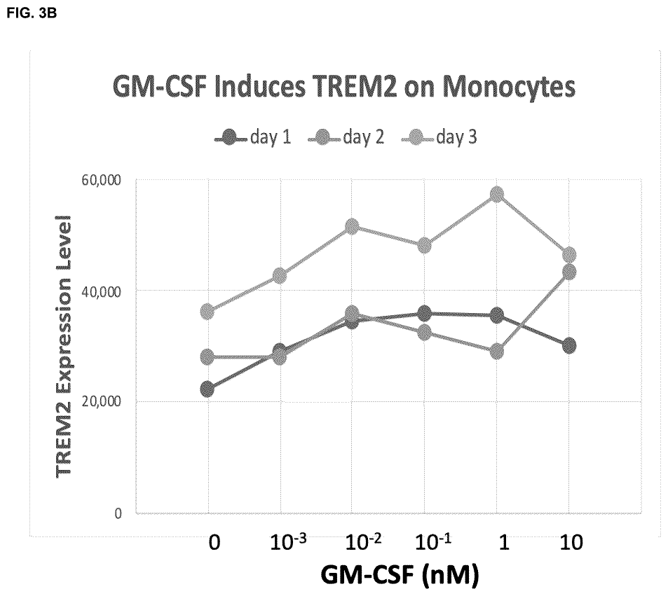

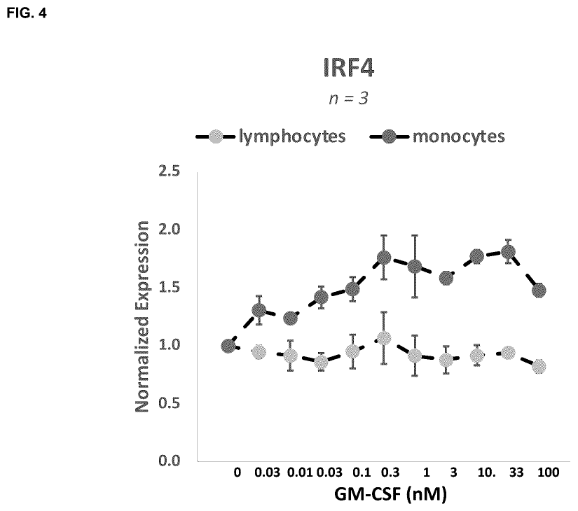

Peripheral blood mononuclear cells from healthy volunteers were cultured overnight in the presence of with sargramostim (LEUKINE) at various concentrations from 0.001 nM to 100 nM. At completion of the culture period, the cells were harvested and stained for the expression of CD26, Musashi-2 (MSI-2) or IRF-4 on monocytes and lymphocytes on day 1. The expression of TREM2 on monocytes and lymphocytes was also measured on days 1, 2 and 3 following treatment. The cells were co-stained for CD14 expression for the identification of monocytes. Expression of the various surface receptors was normalized to the level with no LEUKINE added and plotted versus the varying concentrations of LEUKINE. The procedure was replicated with distinct donors (n=3), and error bars for the expression levels are shown. Treatment with sargramostim (LEUKINE) decreased the expression of CD26 (FIG. 1) and Musashi-2 (MSI-2) (FIG. 2). However, treatment with sargramostim (LEUKINE) increased the expression of TREM2 (FIG. 3A and FIG. 3B) and IRF-4 (FIG. 4) on monocytes but not lymphocytes.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

As used herein, all headings are simply for organization and are not intended to limit the disclosure in any manner. The content of any individual section may be equally applicable to all sections.

What is claimed is:

1. A method of selecting a patient for treatment with an effective amount of a granulocyte-macrophage colony-stimulating factor (GM-CSF) agent for a neurodegenerative or neurological disease or disorder, comprising: determining the presence, absence or amount of Cluster of Differentiation 26 (CD26) in a sample from the patient, wherein the patient is suitable for the treatment if demonstrating an increased or high expression and/or activity of CD26 relative to a pre-treated and/or undiseased state.

2. A method of selecting a patient for treatment with an effective amount of a granulocyte-macrophage colony-stimulating factor (GM-CSF) agent for a neurodegenerative or neurological disease or disorder, comprising: determining the presence, absence or amount of Musashi (MSI) family proteins, optionally selected from Musashi-1 (MSI-1) or Musashi-2 (MSI-2), in a sample from the patient, wherein the patient is suitable for the treatment if demonstrating an increased or high expression and/or activity of one of MSI family proteins, MSI-1 or MSI-2, relative to a pre-treated and/or undiseased state.

3. A method of selecting a patient for treatment with an effective amount of a granulocyte-macrophage colony-stimulating factor (GM-CSF) agent for a neurodegenerative or neurological disease or disorder, comprising: determining the presence, absence or amount of Triggering receptor expressed on myeloid cells 2 (TREM2) in a sample from the patient, wherein the patient is suitable for the treatment if demonstrating a decreased or low expression and/or activity of TREM2 relative to a pre-treated and/or undiseased state.

4. A method of selecting a patient for treatment with an effective amount of a granulocyte-macrophage colony-stimulating factor (GM-CSF) agent for a neurodegenerative or neurological disease or disorder, comprising: determining the presence, absence or amount of Interferon Regulatory Factor 4 (IRF4) in a sample from the patient, wherein the patient is suitable for the treatment if demonstrating a decreased or low expression and/or activity of IRF4 relative to a pre-treated and/or undiseased state.

5. The method of claims 1-4, wherein the patient is treated with an additional agent(s) comprising: administering an effective amount of drug(s) or therapeutics to treat a neurodegenerative or neurological disease or disorder.

6. A method for treating or preventing a neurodegenerative or neurological disease or disorder, comprising:
(a) identifying a patient undergoing or having undergone treatment with an agent for a neurodegenerative or neurological disease or disorder and presenting as failed, intolerant, resistant, or refractory to the treatment with the agent for a neurodegenerative or neurological disease or disorder;
(b) determining the presence, absence or amount of CD26, one or more MSI family proteins, TREM2, and/or IRF4 in a sample from the patient; and
(c) administering an effective amount of a granulocyte-macrophage colony-stimulating factor (GM-CSF) agent to a patient
(i) demonstrating an increased or high expression and/or activity of CD26 and/or MSI family proteins relative to a pre-treated and/or undiseased state; and/or
(ii) demonstrating a decreased or low expression and/or activity of TREM2 and/or IRF4 to a pre-treated and/or undiseased state.

7. The method of claims 1-6, wherein the neurodegenerative or neurological disease or disorder is selected from Alzheimer's disease, Parkinson's disease, progressive supranuclear palsy (PSP), multiple system atrophy (MSA), Lewy body dementia, Parkinson's disease dementia epilepsy, stroke, Huntington's Chorea or Huntington's Disease (HD), cerebral hypoxia, multiple sclerosis, amyotrophic lateral sclerosis (ALS), neovascular glaucoma, optic neuropathy, spinal muscular atrophy (SMA), spinocerebellar ataxia (SCA), and peripheral neuropathy.

8. The method of any of the claims 1-7, wherein the patient is afflicted with a chronic, progressive disorder of the nervous system.

9. The method of claims 1-8, wherein the patient is characterized by having one or more of oxidative stress, loss of neurite integrity, apoptosis, neuronal loss or/and inflammation response, cognitive impairment, cognitive decline, behavioral and personality changes, tremors, bradykinesia, rigidity, impaired posture and balance, loss of automatic movements, decrease in motor coordination, changes in speech, photophobia, difficulty controlling eye muscles, slowed saccadic eye movements, dysphagia, blepharospasm, fainting or lightheadedness due to orthostatic hypotension, dizziness, bladder control problems, well-formed visual hallucinations and delusions, changes in memory, concentration and judgement, memory loss, depression, irritability, anxiety, rapid eye movement (REM) sleep disorder, epileptic seizures, dysesthesia, numbness or tingling, spasticity, difficulty chewing or swallowing, muscle twitching and weakness in a limb, and/or prickling or tingling in feet or hands.

10. The method of any one of claims 1-6, wherein the presence, absence, or amount of CD26, one or more MSI family proteins, TREM2 and/or IRF4 is determined by detection of protein and/or nucleic acids.

11. The method of claim 10, wherein the presence, absence, or amount of CD26, one or more MSI family proteins, TREM2 and/or IRF4 is determined by ELISA, immunohistochemical staining, western blotting, in-cell western, immunofluorescent staining, or fluorescent activating cell sorting (FACS).

12. The method of claims 1-11, wherein the sample is a biological sample which is/or comprises blood, skin sample or tissue sample, plasma, serum, pus, urine, perspiration, tears, mucus, sputum, saliva, cerebrospinal fluid (CSF) and/or other body fluids.

13. The method of any one of claims 1-12, wherein the method prevents, treats, and/or mitigates progression and/or development of the neurodegenerative or neurological disease or disorder in the patient.

14. The method of any one of claims 1-13, wherein the composition elicits a disease-modifying response.

15. The method of any one of claims 1-14, wherein the composition elicits temporarily or permanently slows down cognitive decline.

16. The method of any one of claims 1-13, wherein the composition causes an amelioration of the neurodegenerative or neurological disease or disorder symptoms.

17. The method of any one of claims 1-13, wherein the composition slows the onset and/or development of the neurodegenerative or neurological disease or disorder.

18. The method of any one of the claims 1-17, wherein the method reverses or prevents chronic inflammation in the central nervous system (CNS).

19. The method of claim 18, wherein the method decreases or mitigates the dysfunction of endogenous or exogenous CNS immune cells.

20. The method of claim 17, wherein the method decreases or mitigates the activation of CNS astrocytes and mononuclear phagocytes.

21. The method of claim 20, where the mononuclear phagocytes comprise perivascular macrophages and microglial cells.

22. The method of claims 18-21, wherein the method decreases or mitigates or reverses astrogliopathy.

23. The method of claims 18-22, wherein the method modulates or maintains or supports the glutamine-glutamate balance in the CNS.

24. The method of claims 18-23, wherein the method decreases or mitigates or reverses chronic microglial cell activation.

25. The method of claims 18-24, wherein the method decreases or reverses axonal damage.

26. The method of claims 18-25, wherein the method decreases or prevents excessive production and/or signaling of one or more inflammatory cytokines and/or proteins.

27. The method of claims 1-26, wherein the method decreases or prevents amyloid pathologies.

28. The method of claims 1-26, wherein the method causes a decrease or prevents taupathy.

29. The method of any one of the claims 1-28, wherein the method causes a decrease in the expression and/or activity of the CD26 and/or one or more of MSI family proteins.

30. The method of any one of the claims 1-28, wherein the method causes an increase in the expression and/or activity of the TREM2 and/or IRF4.

31. The method of any one of claims 1-30, wherein the GM-CSF has an amino acid sequence of SEQ ID NO: 1, or a variant of about 90%, or about 93%, or about 95%, or about 97%, or about 98% identity thereto.

32. The method of any one of claims 1-30, wherein the GM-CSF has an amino acid sequence of one of SEQ ID NO: 2, SEQ ID NO: 3, and SEQ ID NO:4, or a variant of about 90%, or about 93%, or about 95%, or about 97%, or about 98% identity thereto.

33. The method of any one of claims 1-32, wherein the GM-CSF is one of molgramostim, sargramostim, and regramostim.

34. The method of claim 33, wherein the GM-CSF is sargramostim.

35. The method of any one of claims 1-34, wherein the GM-CSF is administered at a total dose of about 125 μg, about 150 μg, or about 200 μg, or about 250 μg, or about 300 μg, or about 350 μg.

36. The method of claim 35, wherein the GM-CSF is administered at a total dose of about 250 μg.

37. The method of any one of claim 35 or 36, wherein the GM-CSF is administered at a dosing schedule of once monthly, or twice monthly, or once weekly, or twice weekly, or once daily or twice daily. In embodiments, the GM-CSF is administrated weekly.

38. The method of claim 37, wherein the GM-CSF is sargramostim, administered at a dose of about 125 μg, once weekly.

39. The method of any one of claims 1-38, wherein the GM-CSF is administered to via an intravenous route.

40. The method of any one of claims 1-39, wherein the method further comprises administering one or more additional therapeutic agents, selected from dopamine precursors such as Levodopa, cholinesterase inhibitors such as donepezil (ARICEPT), rivastigmine (EXELON), Galantamine (RAZADYNE), atypical antipsychotics/second generation antipsychotics including serotonin-dopamin antagonists (SDAs), multi-acting receptor-targeted antipsychotics (MARTAS), and D2 partial agonists (e.g. ABILIFY/Aripiprazol), NMDA receptor antagonist memantine, riluzole (RILUTEK), NSAIDs (non-steroidal anti-inflammatory drugs), caffein A2A receptor antagonists and CERE-120 (adeno-associated virus serotype 2-neurturin), deep brain stimulation, TNF-α antagonists including etanercept, adalimumab, infliximab, IFN-γ inhibitors, TGF-β modulators, IL-33 inhibitors, IL-18 inhibitors, VEGF inhibitors, IL-1 inhibitors, inhibitors of pathological beta-amyloid (Aβ) plaques, such as Aβ-directed monoclonal antibodies such as aducanumab (ADUHELM), NSAIDs such as metacetamol and aspirin, anti-diabetic drugs such as linagliptin, suppressors of tau-activation such as liraglutide, miRNA's that target Aβ-plaque formation and tau protein phosphorylation, α-secretase enhancers such as Ginkgo biloba and salvia miltiorrhiza, β-secretase inhibitors such as huanglian and yuanzhi.

41. A method for treating a neurodegenerative or neurological disease or disorder, comprising:
(a) selecting a patient having a neurodegenerative or neurological disease or disorder and one or more of
(i) increased expression and/or activity of CD26 relative to an undiseased state;
(ii) increased expression and/or activity of one or more MSI family proteins relative to an undiseased state;
(iii) decreased expression and/or activity of TREM2 relative to an undiseased state;
(iv) decreased expression and/or activity of IRF4 relative to an undiseased state, and
(b) administering an effective amount of a composition comprising GM-CSF to the patient.

42. The method of claim 41, wherein the method further comprises the step of monitoring the expression and/or activity of CD26 during the course of treatment.

43. The method of claim 42, wherein an increased expression and/or activity of CD26 directs continued administration of GM-CSF.

44. The method of claim 43, wherein decreased expression and/or activity of CD86 directs discontinuation of administration of GM-CSF.

45. The method of claim 41, wherein the method further comprises the step of monitoring the expression and/or activity of one or more Musashi (MSI) family proteins, comprising Musashi-1 and/or Musashi-2, during the course of treatment.

46. The method of claim 45, wherein an increased expression and/or activity of one or more of MSI family proteins directs continued administration of GM-CSF.

47. The method of claim 45, wherein a decreased expression and/or activity of one or more of MSI family proteins directs discontinuation of administration of GM-CSF.

48. The method of claim 41, wherein the method further comprises the step of monitoring the expression and/or activity of TREM2 during the course of treatment.

49. The method of claim 48, wherein a decreased expression and/or activity of TREM2 directs continued administration of GM-CSF.

50. The method of claim 48, wherein an increased expression and/or activity of TREM2 directs discontinuation of administration of GM-CSF.

51. The method of claim 41, wherein the method further comprises the step of monitoring the expression and/or activity of IRF4 during the course of treatment.

52. The method of claim 51, wherein a decreased expression and/or activity of IRF4 directs continued administration of GM-CSF.

53. The method of claim 51, wherein an increased expression and/or activity of IRF4 directs discontinuation of administration of GM-CSF.

54. The method of claims 41-53, wherein the dose of GM-CSF administered to a patient is dependent on the expression and/or activity of CD26 and/or one or more MSI family proteins and/or TREM2 and/or IRF4.

55. The method of any one of claims 41-54, wherein the levels of CD26, MSI family proteins, TREM2 and/or IRF4, are assayed in a biological sample from the patient.

56. The method of claim 55, wherein the biological sample comprises blood, tissue sample, plasma, serum, pus, urine, perspiration, tears, mucus, sputum, saliva, cerebrospinal fluid (CSF) and/or other body fluids.

57. The method of any one of claims 41-56, wherein the neurodegenerative or neurological disease or disorder is one or more of Alzheimer's disease, Parkinson's disease, progressive supranuclear palsy (PSP), multiple system atrophy (MSA), Lewy body dementia, Parkinson's disease dementia epilepsy, stroke, Huntington's Chorea or Huntington's Disease (HD), cerebral hypoxia, multiple sclerosis, amyotrophic lateral sclerosis (ALS), neovascular glaucoma, optic neuropathy, spinal muscular atrophy (SMA), spinocerebellar ataxia (SCA), and peripheral neuropathy.

58. The method of claim 57, wherein the patient is afflicted with Alzheimer's disease or Parkinson's disease.

59. The method of any one of claims 41-58, wherein the patient is characterized by having oxidative stress, loss of neurite integrity, apoptosis, neuronal loss or/and inflammation response, cognitive impairment, cognitive decline, behavioral and personality changes, tremors, bradykinesia, rigidity, impaired posture and balance, loss of automatic movements, decrease in motor coordination, changes in speech, photophobia, difficulty controlling eye muscles, slowed saccadic eye movements, dysphagia, blepharospasm, fainting or lightheadedness due to orthostatic hypotension, dizziness, bladder control problems, well-formed visual hallucinations and delusions, changes in memory, concentration and judgement, memory loss, depression, irritability, anxiety, rapid eye movement (REM) sleep disorder, epileptic seizures, dysesthesia, numbness or tingling, spasticity, difficulty chewing or swallowing, muscle twitching and weakness in a limb, and/or prickling or tingling in feet or hands.

60. The method of any one of claims 41-59, wherein the method prevents, treats, and/or mitigates progression and/or development of the neurodegenerative or neurological disease or disorder.

61. The method of any one of claims 41-60, wherein the method improves the neurodegenerative or neurological disease or disorder in the patient.

62. The method of any one of claims 41-61, wherein the method modulates the expression of one or more cytokines and/or proteins.

63. The method of claim 62, wherein the cytokines and/or proteins are one or more of IL-1, IL-4, IL-6, IL-10, IL-12, IL-18, IL-33, IFN-γ, IP-10, M-CSF, TGF-β, VEGF, and TNFα.

64. The method of any one of claims 41-63, wherein the method causes a decrease in the sequelae of a neurodegenerative or neurological disease or disorder in the patient relative to before treatment.

65. The method of any one of claims 41-64, wherein the GM-CSF has an amino acid sequence of SEQ ID NO: 1, or a variant of about 90%, or about 93%, or about 95%, or about 97%, or about 98% identity thereto.

66. The method of any one of claims 41-65, wherein the GM-CSF has an amino acid sequence of one of SEQ ID NO: 2, SEQ ID NO: 3, and SEQ ID NO: 4, or a variant of about 90%, or about 93%, or about 95%, or about 97%, or about 98% identity thereto.

67. The method of any one of claims 41-66, wherein the GM-CSF is one of molgramostim, sargramostim, and regramostim.

68. The method of claim 67, wherein the GM-CSF is sargramostim.

69. The method of any one of claims 41-68, wherein the GM-CSF is administered at a total dose of about 125 μg, about 150 μg, or about 200 μg, or about 250 μg, or about 300 μg, or about 350 μg.

70. The method of claim 69, wherein the GM-CSF is administered at a total dose of about 250 μg.

71. The method of any one of claims 41-70, wherein the GM-CSF is administered at a dose of about 125 μg, about 150 μg, or about 200 μg, or about 250 μg, or about 300 μg, or about 350 μg.

72. The method of any one of claims 41-71, wherein the GM-CSF is administered at a dosing schedule of once monthly, or twice monthly, or once weekly, or twice weekly, or once daily or twice daily. In embodiments, the GM-CSF is administrated weekly.

73. The method of claim 72, wherein the GM-CSF is sargramostim, administered at a dose of about 125 μg, once weekly.

74. The method of any one of claims 65-73, wherein the GM-CSF is administered via an intravenous route.

75. The method of any one of claims 41-74, wherein the method further comprises administering one or more additional therapeutic agents, selected from dopamine precursors such as Levodopa, cholinesterase inhibitors such as donepezil (ARICEPT), rivastigmine (EXELON), Galantamine (RAZADYNE), atypical antipsychotics/second generation antipsychotics including serotonin-dopamin antagonists (SDAs), multi-acting receptor-targeted antipsychotics (MARTAs), and D2 partial agonists (e.g. ABILIFY/Aripiprazol), NMDA receptor antagonist memantine, riluzole (RILUTEK), NSAIDs (non-steroidal anti-inflammatory drugs), caffein A2A receptor antagonists and CERE-120 (adeno-associated virus serotype 2-neurturin), deep brain stimulation, TNF-α antagonists including etanercept, adalimumab, infliximab, IFN-γ inhibitors, TGF-β modulators, IL-33 inhibitors, IL-18 inhibitors, VEGF inhibitors, IL-1 inhibitors, inhibitors of pathological beta-amyloid (Aβ) plaques, such as Aβ-directed monoclonal antibodies such as aducanumab (ADUHELM), NSAIDs such as metacetamol and asprin, anti-diabetic drugs such as linagliptin, suppressors of tau-activation such as liraglutide, miRNA's that target Aβ-plaque formation and tau protein phosphorylation, α-secretase enhancers such as Ginkgo biloba and salvia miltiorrhiza, β-secretase inhibitors such as huanglian and yuanzhi.


SEQUENCE LISTING (4 sequences)

SEQ ID NO: 1 - protein, 127 aa, Homo sapiens [sequence omitted]

SEQ ID NO: 2 - protein, 127 aa, synthetic construct
APARSPSPSTQPWEHVNAIQEALRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 3 - protein, 127 aa, synthetic construct [sequence omitted]

SEQ ID NO: 4 - protein, 126 aa, synthetic construct
APARSPSPSTQPWEHVNAIQEARRLLNLSRDTAAEMNETVEVISEMFDLQEPTCLQTRLELYKQGLRGSLTKLKGPLTMMASHYKQHCPPTPETSCATQTTFESFKENLKDFLLVIPFDCWEPVQE